(19) United States
(12) Patent Application Publication
(10) Pub. No.: US 2025/0213778 A1
(43) Pub. Date: Jul. 3, 2025

(54) INFUSION PUMP CONTROL SYSTEM AND METHOD FOR MANAGING MULTIPLE INFUSION PUMPS IN A RELAY SEQUENCE

(71) Applicants: BAXTER INTERNATIONAL INC., Deerfield, IL (US); BAXTER HEALTHCARE SA, Glattpark (Opfikon) (CH)

(72) Inventors: Saman Rais Alam Khan, Mumbai (IN); Jelena Leech Rosenberg, Chicago, IL (US); Troy David Heavelyn, Grayslake, IL (US); Andrea F. Ritchie, Corona, CA (US)

(21) Appl. No.: 18/910,957

(22) Filed: Oct. 9, 2024

Related U.S. Application Data
(60) Provisional application No. 63/543,166, filed on Oct. 9, 2023.

(51) Int. Cl. A61M 5/14; A61M 5/168

(52) U.S. Cl. CPC A61M 5/1409; A61M 5/16827; A61M 2205/3331

(57) ABSTRACT

A drug infusion system comprises a plurality of infusion pumps including a first infusion pump and a second infusion pump and a computing device. The computing device comprises a non-transitory computer-readable storage medium and a processor coupled to the non-transitory computer-readable storage medium. The processor is configured to control a plurality of modules to establish a relay sequence of the plurality of infusion pumps to sequentially deliver drugs associated with each of the plurality of infusion pumps to a patient, in response to detecting that the first infusion pump completes an infusion, automatically transfer infusion status information of the first infusion pump to the second infusion pump in the relay sequence, activate the second infusion pump to start infusing in accordance with the relay sequence, remove the first infusion pump from the relay sequence, and retain operational parameters of the first infusion pump.

### PRIORITY CLAIM

This application claims priority to and the benefit as a non-provisional application of U.S. Provisional Patent Application No. 63/543,166, filed Oct. 9, 2024, the entire contents of which are hereby incorporated by reference and relied upon.

### FIELD OF TECHNOLOGY

The present disclosure generally relates to an infusion pump control system and method, and more particularly relates to a system and method for managing multiple infusion pumps in a relay sequence.

### BACKGROUND

A patient's prescribed therapy often requires a controlled drug intake, which may be accomplished by infusing fluidic drugs with an infusion pump. Infusion pumps are medical devices that deliver nutrient or medication fluids such as hormones, antibiotics, chemotherapy drugs, pain relievers, etc. into a patient's body in controlled amounts. There are many types of pumps, including large volume, patient-controlled analgesia, elastomeric, syringe, enteral, and insulin pumps. Some are designed mainly for stationary use at a patient's bedside. Others, called ambulatory infusion pumps, are designed to be portable or wearable.

Clinicians and patients rely on infusion pumps for safe and accurate administration of fluids and medications. However, programming and managing such infusion pumps can be difficult and cumbersome. Programming typically includes preloading a pump program into a pump and then entering pump parameters or data into the pump through a keypad that is directly in the pump. Each time the pump is programmed, pump data must be reentered by a user. Further, managing the status and locations of infusion pumps can be difficult. For example, large volume and syringe pumps may allow users to program continuous infusions, which activate an alarm when a drug reservoir or fluid source container or syringe runs empty. A user must replace the empty fluid container or syringe to continue the infusion or program a different infusion on a pump to continue the therapy. This may result in an interruption of a therapy and alarm fatigue because a user may not be able to respond to the alarm in a timely manner. Once the fluid container or syringe runs empty, the user must first clear the alarm, load a filled fluid container or syringe, reconfirm the program, and then continue the infusion. In some situations, during an infusion that requires multiple fluid containers or syringes to deliver a total volume of a drug sequentially, an active fluid container or syringe may run empty and the user will have to reload a filled fluid container or syringe in order to continue the infusion. Similarly, for infusions that require different drugs to be delivered one after the other, the user has to program the next infusion and load a fluid container or syringe after the prior drug has been delivered. Arranging for a new fluid container or syringe as per drug order, ensuring it is the correct drug and concentration, and then loading the fluid container or syringe may take some time during which the pump is not infusing fluid and the patient may experience discomfort due to interruption in therapy. Additionally, the user has to address multiple pumps with alarms or alerts, thereby increasing the user's workload.

Accordingly, there is a need for a system and method for managing multiple infusion pumps in a relay sequence.

### SUMMARY

Among other features, the present disclosure provides a drug infusion system, comprising: a plurality of infusion pumps including a first infusion pump and a second infusion pump; and a computing device. The computing device may comprise a non-transitory computer-readable storage medium, and a processor coupled to the non-transitory computer-readable storage medium and configured to control a plurality of modules to: establish a relay sequence of the plurality of infusion pumps to sequentially deliver drugs associated with each of the plurality of infusion pumps to a patient; in response to detecting that the first infusion pump completes an infusion, automatically transfer infusion status information of the first infusion pump to the second infusion pump in the relay sequence; activate the second infusion pump to start infusing in accordance with the relay sequence; remove the first infusion pump from the relay sequence; and retain operational parameters of the first infusion pump.

In some embodiments, the processor may be further configured to control the plurality of modules to assign a relay step number for each of the plurality of infusion pumps in order to sequentially deliver the drugs associated with each of the plurality of infusion pumps to the patient. The first infusion pump may be configured to generate and transmit a relay step complete message to remaining infusion pumps of the relay sequence when the first infusion pump completes the infusion.

The processor may be further configured to control the plurality of modules to reprogram the relay step number of the first infusion pump after the first infusion pump completes the infusion, and add the first infusion pump back to the relay sequence after the first infusion pump completes the infusion based at least upon a reprogrammed relay step number of the first infusion pump. The first infusion pump may automatically operate in accordance with the reprogrammed relay step number using the operational parameters after being added back to the relay sequence. In another embodiment, the processor may be configured to control the plurality of modules to generate notification signals after each relay step is completed.

In accordance with other aspects, the present disclosure relates to a computer-implemented method, comprising: establishing a relay sequence of a plurality of infusion pumps to sequentially deliver drugs associated with each of the plurality of infusion pumps to a patient, wherein the plurality of infusion pumps include a first infusion pump and a second infusion pump; in response to detecting that the first infusion pump completes an infusion, automatically transferring infusion status information of the first infusion pump to the second infusion pump in the relay sequence; activating the second infusion pump to start infusing in accordance with the relay sequence; removing the first infusion pump from the relay sequence; and retaining operational parameters of the first infusion pump.

In some embodiments, the computer-implemented method may comprise assigning a relay step number for each of the plurality of infusion pumps in order to sequentially deliver the drugs associated with each of the plurality of infusion pumps to the patient, generating and transmitting, by the first infusion pump, a relay step complete message to remaining infusion pumps of the relay sequence when the first infusion pump completes the infusion.

In yet another embodiment, the computer-implemented method may comprise controlling the plurality of modules to reprogram the relay step number of the first infusion pump after the first infusion pump completes the infusion, and adding the first infusion pump back to the relay sequence after the first infusion pump completes the infusion based at least upon a reprogrammed relay step number of the first infusion pump. The computer-implemented method may further comprise automatically operating the first infusion pump in accordance with the reprogrammed relay step number using the operational parameters after being added back to the relay sequence, and generating notification signals after each relay step is completed.

In accordance with additional aspects, the present disclosure relates to a non-transitory computer readable medium storing computer executable instructions for a system, the instructions being configured for: establishing, by a processor of a computing device, a relay sequence of a plurality of infusion pumps to sequentially deliver drugs associated with each of the plurality of infusion pumps to a patient, wherein the plurality of infusion pumps include a first infusion pump and a second infusion pump; in response to detecting that the first infusion pump completes an infusion, automatically transferring infusion status information of the first infusion pump to the second infusion pump in the relay sequence; activating the second infusion pump to start infusing in accordance with the relay sequence; removing the first infusion pump from the relay sequence; and retaining operational parameters of the first infusion pump.

In one embodiment, the non-transitory computer readable medium may comprise instructions for: assigning a relay step number for each of the plurality of infusion pumps in order to sequentially deliver the drugs associated with each of the plurality of infusion pumps to the patient; and generating notification signals after each relay step is completed.

In yet another embodiment, the non-transitory computer readable medium may comprise instructions for generating and transmitting, by the first infusion pump, a relay step complete message to remaining infusion pumps of the relay sequence when the first infusion pump completes the infusion, and reprogramming the relay step number of the first infusion pump after the first infusion pump completes the infusion.

Moreover, the non-transitory computer readable medium may comprise instructions for adding the first infusion pump back to the relay sequence after the first infusion pump completes the infusion based at least upon a reprogrammed relay step number of the first infusion pump, and automatically operating the first infusion pump in accordance with the reprogrammed relay step number using the operational parameters after being added back to the relay sequence.

In accordance with other aspects, the present disclosure relates to a drug infusion system, comprising: a plurality of infusion pumps including a first infusion pump and a second infusion pump; and a computing device. The computing device may comprise a non-transitory computer-readable storage medium; and a processor coupled to the non-transitory computer-readable storage medium and configured to control a plurality of modules to: establish a relay sequence of the plurality of infusion pumps to sequentially deliver drugs associated with each of the plurality of infusion pumps to a patient; in response to detecting that the first infusion pump completes an infusion, automatically transfer operational parameters of the first infusion pump to the second infusion pump in the relay sequence; activate the second infusion pump to start infusing in accordance with the relay sequence based on the operational parameters of the first infusion pump; remove the first infusion pump from the relay sequence; and retain the operational parameters of the first infusion pump.

In some embodiments, the processor may be configured to control the plurality of modules to assign a relay step number for each of the plurality of infusion pumps in order to sequentially deliver the drugs associated with each of the plurality of infusion pumps to the patient. The plurality of infusion pumps of the relay sequence may have a same care area, drug and concentration.

The processor of the computing device may be further configured to control the plurality of modules to: reprogram the relay step number of the first infusion pump after the first infusion pump completes the infusion; add the first infusion pump back to the relay sequence after the first infusion pump completes the infusion based at least upon a reprogrammed relay step number of the first infusion pump; and generate notification signals after each relay step is completed. The first infusion pump may automatically operate in accordance with the reprogrammed relay step number using the operational parameters after being added back to the relay sequence.

In some implementations, the processor may be further configured to control the plurality of modules to perform a plurality of logic checks of each infusion pump before establishing the relay sequence of the plurality of infusion pumps, including: determining whether a type of a pump to be added is same as the first or the second infusion pumps, determining whether the relay sequence is programmed on at least one infusion pump, determining whether the pump to be added to the relay sequence is loaded with an identical software version as the first or the second infusion pumps, determining whether the pump to be added is loaded with an identical drug library as the first or the second infusion pumps, determining whether the pump to be added has an identical care area as the first or the second infusion pumps, determining whether the pump to be added has an identical drug as the first or the second infusion pumps, determining whether the pump to be added has an identical concentration as the first or the second infusion pumps, determining whether the pump to be added is programmed with an identical medical coding modifier as the first or the second infusion pumps, and determining whether the pump to be added is loaded with an identical syringe size and manufacturer as the first or the second infusion pumps.

In certain embodiments, the operational parameters of the first infusion pump may include a flow rate of a drug, a dose of the drug, an amount of the drug, patient weight information, a patient's body surface area, occlusion setting, alarm volume setting, phase complete setting, pressure indicator setting, amount given, auto-brightness setting, brightness setting, keypad backlight setting, air in line setting, and near empty setting.

In accordance with further aspects, the present disclosure relates to a computer-implemented method, comprising: establishing a relay sequence of a plurality of infusion pumps to sequentially deliver drugs associated with each of the plurality of infusion pumps to a patient, wherein the plurality of infusion pumps include a first infusion pump and a second infusion pump; in response to detecting that the first infusion pump completes an infusion, automatically transferring operational parameters of the first infusion pump to the second infusion pump in the relay sequence; activating the second infusion pump to start infusing in accordance with the relay sequence based on the operational parameters of the first infusion pump; removing the first infusion pump from the relay sequence; and retaining the operational parameters of the first infusion pump.

In one embodiment, the computer-implemented method may further comprise assigning a relay step number for each of the plurality of infusion pumps in order to sequentially deliver the drugs associated with each of the plurality of infusion pumps to the patient. The plurality of infusion pumps of the relay sequence may have a same care area, drug and concentration.

Moreover, the computer-implemented method may further comprise reprogramming the relay step number of the first infusion pump after the first infusion pump completes the infusion; adding the first infusion pump back to the relay sequence after the first infusion pump completes the infusion based at least upon a reprogrammed relay step number of the first infusion pump; and generating notification signals after each relay step is completed.

In some embodiments, the computer-implemented method may comprise automatically operating the first infusion pump in accordance with the reprogrammed relay step number using the operational parameters after being added back to the relay sequence.

In certain implementations, the computer-implemented method may comprise performing a plurality of logic checks of each infusion pump before establishing the relay sequence of the plurality of infusion pumps, including: determining whether a type of a pump to be added is same as the first or the second infusion pumps, determining whether the relay sequence is programmed on at least one infusion pump, determining whether the pump to be added to the relay sequence is loaded with an identical software version as the first or the second infusion pumps, determining whether the pump to be added is loaded with an identical drug library as the first or the second infusion pumps, determining whether the pump to be added has an identical care area as the first or the second infusion pumps, determining whether the pump to be added has an identical drug as the first or the second infusion pumps, determining whether the pump to be added has an identical concentration as the first or the second infusion pumps, determining whether the pump to be added is programmed with an identical medical coding modifier as the first or the second infusion pumps, and determining whether the pump to be added is loaded with an identical syringe size and manufacturer as the first or the second infusion pumps.

In certain embodiments, the operational parameters of the first infusion pump include a flow rate of a drug, a dose of the drug, an amount of the drug, patient weight information, a patient's body surface area, occlusion setting, alarm volume setting, phase complete setting, pressure indicator setting, amount given, auto-brightness setting, brightness setting, keypad backlight setting, air in line setting, and near empty setting.

In yet another aspect, the present disclosure relates to a non-transitory computer readable medium storing computer executable instructions for a system, the instructions being configured for: establishing, by a processor of a computing device, a relay sequence of a plurality of infusion pumps to sequentially deliver drugs associated with each of the plurality of infusion pumps to a patient, wherein the plurality of infusion pumps include a first infusion pump and a second infusion pump; in response to detecting that the first infusion pump completes an infusion, automatically transferring operational parameters of the first infusion pump to the second infusion pump in the relay sequence; activating the second infusion pump to start infusing in accordance with the relay sequence based on the operational parameters of the first infusion pump; removing the first infusion pump from the relay sequence; and retaining the operational parameters of the first infusion pump.

In one embodiment, the non-transitory computer readable medium may further comprise instructions for assigning a relay step number for each of the plurality of infusion pumps in order to sequentially deliver the drugs associated with each of the plurality of infusion pumps to the patient; reprogramming the relay step number of the first infusion pump after the first infusion pump completes the infusion; adding the first infusion pump back to the relay sequence after the first infusion pump completes the infusion based at least upon a reprogrammed relay step number of the first infusion pump; and generating notification signals after each relay step is completed. The plurality of infusion pumps of the relay sequence may have a same care area, drug and concentration.

In another embodiment, the non-transitory computer readable medium further comprises instructions for automatically operating the first infusion pump in accordance with the reprogrammed relay step number using the operational parameters after being added back to the relay sequence.

Moreover, the non-transitory computer readable medium further comprises instructions for performing a plurality of logic checks of each infusion pump before establishing the relay sequence of the plurality of infusion pumps, including: determining whether a type of a pump to be added is same as the first or the second infusion pumps, determining whether the relay sequence is programmed on at least one infusion pump, determining whether the pump to be added to the relay sequence is loaded with an identical software version as the first or the second infusion pumps, determining whether the pump to be added is loaded with an identical drug library as the first or the second infusion pumps, determining whether the pump to be added has an identical care area as the first or the second infusion pumps, determining whether the pump to be added has an identical drug as the first or the second infusion pumps, determining whether the pump to be added has an identical concentration as the first or the second infusion pumps, determining whether the pump to be added is programmed with an identical medical coding modifier as the first or the second infusion pumps, and determining whether the pump to be added is loaded with an identical syringe size and manufacturer as the first or the second infusion pumps.

In some implementations, the operational parameters of the first infusion pump may include a flow rate of a drug, a dose of the drug, an amount of the drug, patient weight information, a patient's body surface area, occlusion setting, alarm volume setting, phase complete setting, pressure indicator setting, amount given, auto-brightness setting, brightness setting, keypad backlight setting, air in line setting, and near empty setting.

The above simplified summary of example aspects serves to provide a basic understanding of the present disclosure. This summary is not an extensive overview of all contemplated aspects, and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplary pointed out in the claims.

### BRIEF DESCRIPTION OF THE DRAWINGS

The accompanying drawings, which are incorporated into and constitute a part of this specification, illustrate one or more example aspects of the present disclosure and, together with the detailed description, serve to explain their principles and implementations.

FIG. 1 illustrates an exemplary architecture for managing and controlling multiple infusion pumps in a relay sequence, according to an exemplary aspect of the present disclosure;

FIGS. 2 and 3 respectively illustrate how to program infusion pumps in a relay sequence with sequentially assigned relay step numbers on a user interface, according to an exemplary aspect of the present disclosure;

FIG. 4 illustrates another exemplary architecture for managing and controlling multiple infusion pumps in a relay sequence for automatically transferring infusion parameters of a completed pump to a subsequent pump in a relay sequence, according to an exemplary aspect of the present disclosure;

FIG. 5 illustrates how to program an infusion pump in a relay sequence to activate a take-over mode on a user interface, according to an exemplary aspect of the present disclosure;

FIG. 6 illustrates a flowchart a computer-implemented method for sequentially delivering drugs associated with each of a plurality of infusion pumps to a patient in a relay arrangement, according to an exemplary aspect of the present disclosure; and

FIG. 7 illustrates a flowchart of a computer-implemented method for sequentially delivering drugs associated with each of a plurality of infusion pumps to a patient by automatically transferring infusion parameters of a completed infusion pump to a subsequent infusion pump, according to an exemplary aspect of the present disclosure.

### DETAILED DESCRIPTION

Various aspects of present disclosure will be described with reference to the drawings, wherein like reference numerals are used to refer to like elements throughout. In the following description, for purposes of explanation, numerous specific details are set forth in order to promote a thorough understanding of one or more aspects of the present disclosure. It may be evident in some or all instances, however, that any aspects described below can be practiced without adopting the specific design details described below.

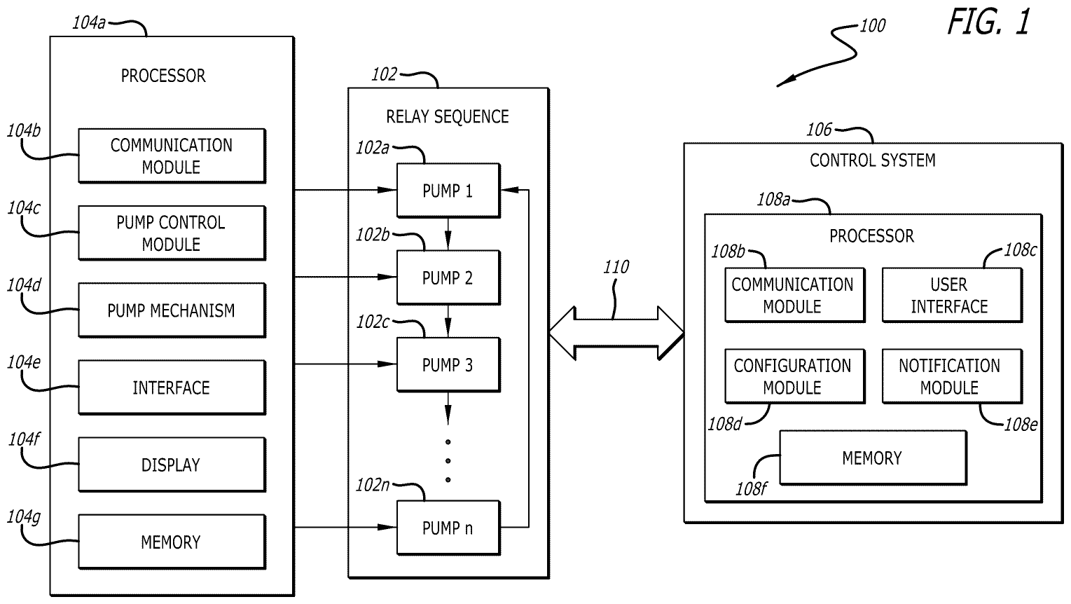

In accordance with aspects of the present disclosure, FIG. 1 is a systematic diagram illustrating an exemplary architecture 100 for managing and controlling multiple infusion pumps in a relay sequence 102. In one embodiment, a control system 106 may be configured to operate and manage at least a portion of a plurality of medication infusion pumps 102a, 102b, 102c . . . 102n to provide an auto-continuous infusion therapy sequentially to a patient. The architecture 100 may allow a user to program a relay step on a selected infusion pump 102a, 102b, 102c . . . 102n of the relay sequence 102, reprogram a completed pump, and add the completed pump to the end of a relay infusion. The control system 106 may be configured to maintain the programmed parameters of the completed pump, such that the user will only have to program the relay step to add it back to the relay sequence 102, thereby minimizing user inputs on a specific pump. As a result, a loop of relay steps may be created by reusing pumps as new relay steps. This at least ensures that a limited number of pumps are required per infusion. In other words, the number of pumps per relay infusion may be less than the number of relay steps. For example, a relay infusion with 20 relay steps does not need 20 different pumps.

In one embodiment, the control system 106 of the present disclosure may include at least one processor 108a configured to control and execute a plurality of modules including, but not limited to, a communication module 108b, a user interface 108c, a configuration module 108d, and a notification module 108e. Memory 108f, which is a non-transitory machine readable medium coupled to the processor 108a, may be configured to store at least information related to each pump unit 102a, 102b, 102c . . . 102n and/or the relay sequence 102, and at least one set of data structures or instructions (e.g., software) embodying or utilized by at least one of the techniques or functions described herein. On the other hand, each pump unit 102a, 102b, 102c . . . 102n of the relay sequence 102 may include at least one processor 104a configured to control and execute a plurality of modules including, but not limited to, a communication module 104b, a pump control module 104c, pump mechanism 104d, an interface 104e, and a display 104f. Memory 104g, which is a non-transitory machine readable medium coupled to the processor 104a of each pump unit, may be configured to store at least information related to each pump unit 102a, 102b, 102c . . . 102n and at least one set of data structures or instructions (e.g., software) embodying or utilized by at least one of the techniques or functions described herein.

The term “module” as used herein refers to a real-world device, component, or arrangement of components and circuitries implemented using hardware, such as by an application specific integrated circuit (ASIC) or field-programmable gate array (FPGA), for example, or as a combination of hardware and software, such as by a microprocessor system and a set of instructions to implement the module's functionality, which (while being executed) transform the microprocessor system into a special purpose device. A module may also be implemented as a combination of the two, with certain functions facilitated by hardware alone, and other functions facilitated by a combination of hardware and software. Each module may be realized in a variety of suitable configurations, and should not be limited to any example implementation exemplified herein.

It should be appreciated that the term “non-transitory machine readable medium” may include a single medium or multiple media (e.g., one or more caches) configured to store the at least one instruction. The term “machine readable medium” may include any medium that is capable of storing, encoding, or carrying instructions for execution by all modules of a corresponding computing device and that cause these modules to perform at least one of the techniques of the present disclosure, or that is capable of storing, encoding or carrying data structures used by or associated with such instructions. Non-limiting machine readable medium examples may include solid-state memories, and optical and magnetic media. Specific examples of machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); Solid State Drives (SSD); and CD-ROM and DVD-ROM disks.

The control system 106 and the relay sequence 102 formed by at least a portion of the pump unit 102a, 102b, 102c . . . 102n may be communicably coupled through any suitable interface 110. In one embodiment, the interface 110 may facilitate wireless, wired, contact, near proximity, or contactless communication between the communication module 108b of the control system 106 and the communication module 104b of the relay sequence 102. While illustrated as a single or continuous interface, the interface 110 may be logically divided into individual interfaces on either the control system 106 or the relay sequence 102 without departing from the scope of this disclosure, so long as at least a portion of the interface 110 may facilitate communications between the control system 106 and the relay sequence 102.

In some implementations, the communication module 108b of the control system 106 may be configured to obtain one or more user instructions for controlling at least one of the pump unit 102a, 102b, 102c . . . 102n of the relay sequence 102 via, e.g., the user interface 108c, an infrared port, a universal serial bus (USB) port, an RFID reader, a computing device of a user (e.g., a smart phone or device), a barcode reader, or any suitable devices. For example, the communication module 108b may process, translate, and transmit user entered data, calculation data, modification data, operating instructions, and the like to the corresponding communication module 104b of a specific pump unit 102a, 102b, 102c . . . 102n of the relay sequence 102. The processor 104a of each pump unit may process the received information in order to carry out any instructions or user modifications required by the control system 106.

The user interface 108c of the control system 106 may be configured to interact with a user to manage a selected infusion pump 102a, 102b, 102c . . . 102n and/or the relay sequence 102 and display system information to the user. In some implementations, the user interface 108c may provide guidance and recommendations to the user when a system function is scheduled, or when the user has requested a system modification. For example, the user interface 108c may provide suggestions and calculation values to assist the user in determining a course of action regarding an ongoing auto-continuous infusion therapy.

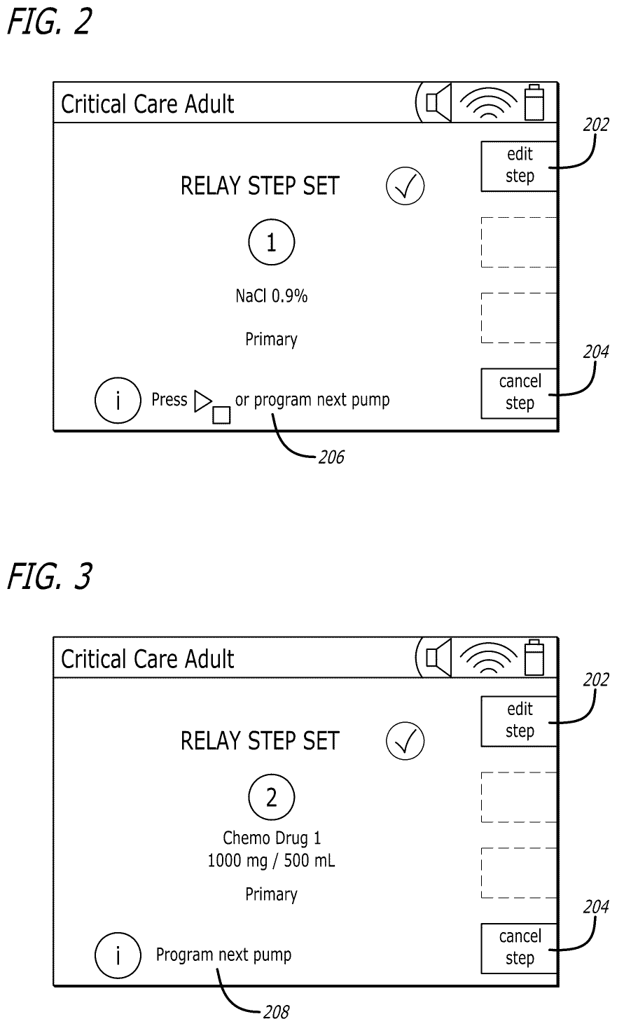

In one aspect, the configuration module 108d of the control system 106 may allow a user to program a relay step on a selected infusion pump 102a, 102b, 102c . . . 102n of the relay sequence 102 via the user interface 108c, such that the relay sequence 102 automatically starts when an active pump of the relay sequence 102 completes an infusion. That is, a series of pumps 102a, 102b, 102c . . . 102n may be assigned step numbers and are configured to deliver drug one after the other with minimal user interaction for transition from one step to another. Further, the configuration module 108d may allow a user to pre-program multiple steps on different pumps which in turn converts the individual pump infusions into one large relay sequence. Referring to FIGS. 2 and 3, at least two infusion pumps may be set up in a relay by the configuration module 108d: sodium chloride (NaCl) 0.9% intravenous infusion as relay step 1, and chemo drug 1 (1000 mg/500 ml) as relay step 2. For each pump, the configuration module 108d may further allow the user to edit the relay step 202 and/or cancel the relay step 204 via the user interface 108c. As shown in FIG. 2, the user may start or stop 206 a specific infusion pump or program the next pump in the relay sequence 102. For a scheduled pump in the relay sequence 102, as shown in FIG. 3, the user may program 208 next pump by, e.g., adding a new pump or a completed pump to the end of a relay infusion. As will be described fully below, the present disclosure may maintain and store all programmed parameters of a completed pump, such that the user may add it back to the relay sequence 102 by reprograming its prior relay step number without inputting all the operational parameters. As a result, a loop of relay steps may be created by reusing pumps as new relay steps.

In some implementations, the series of pumps 102a, 102b, 102c . . . 102n may be mounted on a HUB that allows communications therebetween via a controller area network (CAN) bus. For example, the relay sequence 102 may include at least two self-contained, independently operable syringe pumps coupled in a relay arrangement for a controlled administration of fluids such as pharmaceutical drugs, parenteral nutrition, blood and blood products and enteral nutrition. Each pump may be configured to deliver an infusion through any clinically accepted routes of administration such as intravenous, arterial, enteral, and subcutaneous. Each pump may be operated via an interface 104e through e.g., a touch screen, keyboard, any suitable information entry device, or a data collection device that can be connected to the interface 104e of each pump. In addition, each pump may include a display 104f including but not limited to light emitting diodes (LEDs), touch screens, liquid crystal display (LCD) screens, or other visual devices. Audio indicators may be also included in each pump such as buzzer chips, piezoelectric indicators, or other suitable technology.

In some aspects, the processor 104a of a selected pump may be configured to receive instructions from the configuration module 108d of the control system 106 and direct the pump control module 104c and pump mechanism 104d to discharge fluid from a corresponding fluid container associated with the selected pump in accordance with the instructions. For example, the pump mechanism 104 may include one or more pressure sensors configured to monitor and measure fluid pressure at various locations (e.g., extension tubes, couplers, connectors, valves, and reflux barriers) of a syringe pump. Based at least upon the feedback in terms of fluid pressure provided by the pump mechanism 104d, the pump control module 104c may be configured to compensate for changes in the elevation of an active syringe pump relative to the patient and for frictional and other mechanical variations from one syringe pump to the next in the relay sequence 102. For example, the pump control module 104c may restore fluid pressure in response to detecting a pressure decrease from one pump exceeding a predetermined threshold value in order to avoid an interruption in fluid administration. A subsequent syringe pump in the relay sequence 102 may be controlled by the pump control module 104c to run in a forward direction automatically either in response to sensing a pressure drop occurring during relay or in response to signals transmitted by a prior infusion pump over the CAN bus. In one embodiment, the subsequent syringe pump may be configured to move in the forward direction at a rate of speed that is higher than the normal rate of speed consistent with infusion parameters programmed by the user into the syringe pump in order to compensate for the lost fluid pressure. In response to detecting that the fluid pressure in the extension tubes has been restored to a reference value via the pressure sensors of the pump mechanism 104, the subsequent syringe pump may be controlled by the pump control module 104c to operate at a normal rate of speed, thereby providing a steady automated changeover.

In accordance with aspects of the present disclosure, the processor 104a of each pump of the relay sequence 102 may be configured to control the pump control module 104c to transfer infusion status information to other pumps via the CAN bus and vice versa. When an active pump of the relay sequence 102 completes an infusion, the pump control module 104c of the active pump may broadcast a relay step complete message to all other pumps. As a result, the active pump broadcasts its infusion status parameters, and the next pump in the relay sequence 102 is automatically triggered to start infusing. After the infusion on a relay step is completed, that pump is removed from the relay sequence 102, but the infusion parameters are retained either on memory 104g associated with each pump or memory 108f of the control system 106.

Referring back to FIG. 1, 3 pumps may be programmed for a relay infusion as steps 1, 2 and 3. Once relay step 1 (pump 1 102a) completes infusion and transfers infusion status information to the next pump (pump 2 102b), it is removed out of the relay sequence 102. Pump 1 may be programmed as relay step 4. Similarly, when relay step 2 (pump 2 102b) is completed and is out of the relay sequence 102, pump 2 may be added back to the relay sequence 102 as relay step 5. Operational infusion parameters of a specific pump that has been removed from the relay sequence 102 may be retained, such that the pump can be readily added back to the relay sequence 102 based on a pre-programmed relay step number.

In yet another aspect, the control system 106 of the present disclosure may generate alarms specific to interruption of the relay sequence 102, thereby mitigating risks of interruption in a therapy. For example, each pump in the relay sequence 102 may be configured to denote a relay step number at all times, thereby being uniquely identifiable as a pump in relay sequence 102. When an active standard relay step completes an infusion and at least one inactive relay step is programmed in the relay sequence 102, the notification module 108e of the control system 106 may be configured to display a relay step completed alert on a target pump and perform the following: generate a relay step complete audio tone, notify the user that a current relay step infusion has completed, and display the relay step number, care area, drug and concentration, thereby allowing the user to review the completed relay step infusion information. In one embodiment, the notification module 108e may be configured to generate at least one of a visual message, an audio alert, vibration, and/or other available alerting mechanism on either the user interface 108c or a user's smartphone, tablet or other electronic devices connected with the control system 106 and associated with the user.

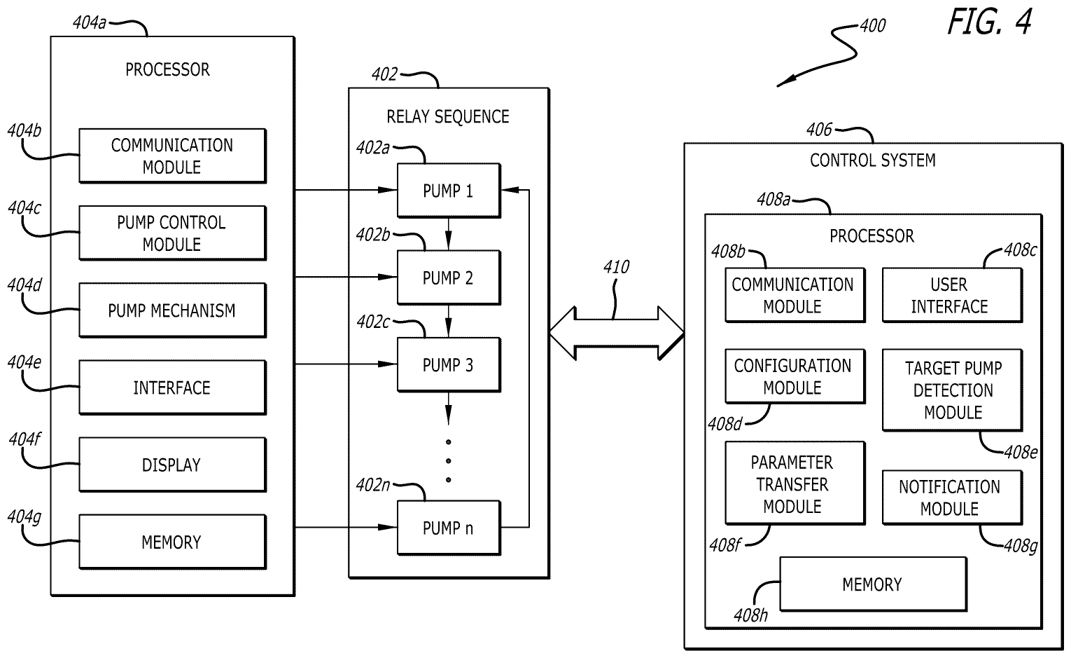

In some circumstances, there may be a clinical need to infuse the same drug to a patient continuously for days or weeks. However, reprogramming the same infusion on multiple pumps may be tedious and prone to errors. In accordance with additional aspects of the present disclosure, FIG. 4 is a systematic diagram illustrating an exemplary architecture 400 for automatically transferring infusion parameters among multiple infusion pumps in a relay sequence 402. In one embodiment, a control system 406 of the present disclosure may include at least one processor 408a configured to control and execute a plurality of modules including, but not limited to, a communication module 408b, a user interface 408c, a configuration module 408d, a target pump detection module 408e, a parameter transfer module 408f, and a notification module 408g. Memory 408h, which is a non-transitory machine readable medium coupled to the processor 408a, may be configured to store at least information related to each pump unit 402a, 402b, 402c . . . 402n and/or the relay sequence 402, and at least one set of data structures or instructions (e.g., software) embodying or utilized by at least one of the techniques or functions described herein. On the other hand, similar to the relay sequence 102 of FIG. 1, each pump unit 402a, 402b, 402c . . . 402n of the relay sequence 402 may include at least one processor 404a configured to control and execute a plurality of modules including, but not limited to, a communication module 404b, a pump control module 404c, pump mechanism 404d, an interface 404e, and a display 404e. Memory 404g, which is a non-transitory machine readable medium coupled to the processor 404a of each pump unit, may be configured to store at least information related to each pump unit 402a, 402b, 402c . . . 402n and at least one set of data structures or instructions (e.g., software) embodying or utilized by at least one of the techniques or functions described herein.

The control system 406 and the relay sequence 402 formed by at least a portion of the pump unit 402a, 402b, 402c . . . 402n may be communicably coupled through any suitable interface 410. In one embodiment, the interface 410 may facilitate wireless, wired, contact, near proximity, or contactless communication between the communication module 408b of the control system 406 and the communication module 404b of the relay sequence 402. While illustrated as a single or continuous interface, the interface 410 may be logically divided into individual interfaces on either the control system 406 or the relay sequence 402 without departing from the scope of this disclosure, so long as at least a portion of the interface 410 may facilitate communications between the control system 406 and the relay sequence 402.

In some implementations, the communication module 408b of the control system 406 may be configured to obtain one or more user instructions for automatically transferring infusion parameters between two or more pump unit 402a, 402b, 402c . . . 402n of the relay sequence 402 via, e.g., the user interface 408c, an infrared port, a USB port, an RFID reader, a computing device of a user (e.g., a smart phone or device), a barcode reader, or any suitable devices. For example, the communication module 408b may process, translate, and transmit user entered data, calculation data, modification data, operating instructions, and the like to the corresponding communication module 404b of a specific pump unit 402a, 402b, 402c . . . 402n of the relay sequence 402. The processor 404a of each pump unit may process the received information in order to carry out any instructions or user modifications required by the control system 406.

The user interface 408c of the control system 406 may be configured to interact with a user to manage a selected infusion pump 402a, 402b, 402c . . . 402n and/or the relay sequence 402 and display system information to the user. In some implementations, the user interface 408c may provide guidance and recommendations to the user when a system function is scheduled, or when the user has requested a system modification. For example, the user interface 408c may be configured to provide suggestions and calculation values to assist the user in determining a course of action regarding an ongoing auto-continuous infusion therapy.

In one aspect, the configuration module 408d of the control system 406 may allow a user to program and assign step numbers to at least a portion of a plurality of medication infusion pumps 402a, 402b, 402c . . . 402n forming the relay sequence 402 with the same care area, drug and concentration before starting an auto-continuous infusion therapy to a patient. With respect to each pump, similar to the systematic setup disclosed above in relation to FIG. 1, the configuration module 408d may further allow the user to edit the relay step and/or cancel the relay step via the user interface module 408c. For example, the user may start or stop a specific infusion pump or program the next pump in the relay sequence 402. For a scheduled pump in the relay sequence 402, the user may program next pump by, e.g., adding a new pump or a completed pump to the end of a relay infusion. The present disclosure may maintain and store all programmed parameters of a completed pump, such that the user may add it back to the relay sequence 402 without inputting all the parameters. As a result, a loop of relay steps may be created by reusing pumps as new relay steps.

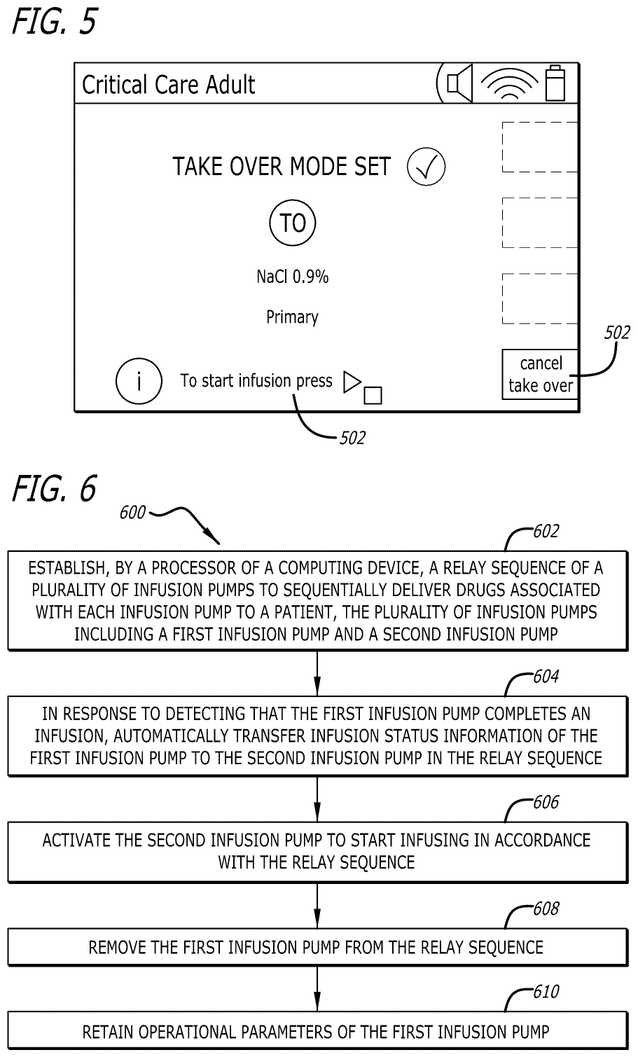

In certain aspects, to activate a “take-over” mode on a series of infusion pumps with the same care area, drug and concentration, the configuration module 408d may require a user program all pertinent infusion parameters on a first pump of the relay sequence 402 only. Referring to FIG. 5, the user may add 502 or remove 504 an infusion pump containing sodium chloride (NaCl) 0.9% intravenous infusion to the relay sequence 402 via the “take over mode set” feature on the user interface 408c. One or more pumps having the same drug, concentration and care area may be added to the relay sequence 402 even after the infusion has started. A series of pumps can be assigned step numbers and will deliver the same drug one after the other with minimal user interaction for transition from one relay step to another. This allows the user to pre-program multiple steps on different pumps which in turn converts the individual pump infusions into one large relay sequence. As such, the control system 406 of the present disclosure provides the flexibility to pre-program an infusion therapy, allows a user to leave the bedside during the infusion, and ensures that the patient receives correct drugs with minimum interruption.

In a preferred implementation, the series of pumps 402a, 402b, 402c . . . 402n may be mounted on a HUB that allows communication via a CAN bus. For example, the relay sequence 402 may include at least two self-contained, independently operable syringe pumps coupled in a relay arrangement for the controlled administration of fluids such as pharmaceutical drugs, parenteral nutrition, blood and blood products and enteral nutrition. Each pump may be configured to deliver an infusion through any clinically accepted routes of administration such as intravenous, arterial, enteral, and subcutaneous. Each pump may be operated via an interface 404e through e.g., a touch screen, keyboard, any suitable manual entry device, or a data collection device that can be connected to the interface 404e of each pump. In addition, each pump may include a display 404f including but not limited to LEDs, touch screens, LCD screens, or other visual devices. Audio indicators may be also included in each pump such as buzzer chips, piezoelectric indicators, or other suitable technology. The processor 404a of a selected pump may be configured to receive instructions from various modules of the control system 406 and control the pump control module 404c and pump mechanism 404d to discharge fluid from a corresponding fluid container associated with the selected pump in accordance with the instructions. For example, the pump mechanism 404 may include one or more pressure sensors configured to monitor and measure fluid pressure at various locations (e.g., extension tubes, couplers, connectors, valves, and reflux barriers) of a syringe pump. Based at least upon the feedback in terms of fluid pressure provided by the pump mechanism 404d, the pump control module 404c may be configured to compensate for changes in the elevation of an active syringe pump relative to the patient and for frictional and other mechanical variations from one syringe pump to the next in the relay sequence 402. For example, the pump control module 404c may restore fluid pressure in response to detecting a pressure decrease from one pump exceeding a predetermined threshold value in order to avoid an interruption in fluid administration. A subsequent syringe pump in the relay sequence 402 may be controlled by the pump control module 404c to run in a forward direction automatically either in response to sensing a pressure drop occurring during relay or in response to signals transmitted by a prior infusion pump over the CAN bus. In one embodiment, the subsequent syringe pump may be configured to move in the forward direction at a rate of speed that is higher than the normal rate of speed consistent with infusion parameters programmed by the user into the syringe pump in order to compensate for the lost pressure. In response to detecting that the fluid pressure in the extension tubes has been restored to a reference value via the pressure sensors of the pump mechanism 404, the subsequent syringe pump may be controlled by the pump control module 404c to operate at a normal rate of speed, thereby providing a steady automated changeover.

One of the objectives of the control system 406 is to automatically start infusion on the next step of the relay sequence 402 when an active pump completes an infusion and transfer the infusion parameters to next pump. The next pump will then deliver the infusion at the same rate as the previous pump. Thus, every pump in the relay sequence 402 mirrors the infusion parameters of the previous pump. The user may only need to program all the required parameters like dose/rate etc. on the first pump of the relay sequence 402. All other pumps in the relay sequence 402 will be programmed with the same care area, drug and concentration and added to the sequence via the “take over mode set” feature. Specifically, in response to detecting a target pump having the same care area, drug and concentration, the control system 406 may be configured to prompt the user to add the target pump to the take-over relay sequence. As such, multiple pumps may be added to the relay sequence 402 with reduced user inputs for programming identical parameters on individual pumps.

In some implementations, since a series of pumps are mounted on a HUB that allows communications therebetween via the CAN bus, each pump can transfer infusion status information to other pumps and vice versa. When an active pump of the relay sequence 402 completes an infusion, the pump control module 404c of the active pump may broadcast a relay step complete message to all other pumps and start transferring the program parameters to next relay step only, thereby triggering the next pump in the take-over sequence 402 to start infusing at the same rate as the previous pump. In some embodiments, the user may edit the parameters of an actively infusing pump. The parameter transfer module 408f of the control system 406 may be configured to transfer the updated parameters to the next pump. After the infusion on a relay step is completed, that pump may be removed from the relay sequence 402, but the infusion parameters may be stored either on memory 404g associated with each pump or memory 408f of the control system 406. The user may then reprogram the relay step on the completed pump and add it to the end of the relay infusion. The control system 406 may maintain the programmed parameters on the completed pump, such that the user will only have to program the relay step to add it back to the relay sequence 402, thereby minimizing user inputs on a specific pump. As a result, a loop of relay steps may be created by reusing pumps as new relay steps. This at least ensures that a limited number of pumps are required per infusion. In other words, the number of pumps per relay infusion may be less than the number of relay steps. For example, a relay infusion with 20 relay steps does not need 20 different pumps.

In some implementations, direct communication from pump to pump in a relay arrangement may enable a user to transfer infusion related information, pump operating history, user information, configuration parameters, and other information directly form one pump unit to another. Such a transfer does not require any additional equipment or setup. By contrast, an approach that involves, for example, uploading and downloading from a local or remote database server system may require a communication connection, access to the specific data storage site, and additional hardware. The user may not have access to these setups when a pump unit begins to fail. Direct pump to pump data communication may enable relevant information to be received in a new pump unit, while still retaining information and history. For example, direct pump information transfer may transfer infusion related information, identification, configuration, device setting information, operating history, historical dosage, and consumption information from pump to pump, resulting in an easy and direct initialization of subsequent pump units in the relay sequence 402. Thus, the user may replace a completed pump, but have all the relevant information of the completed pump available in the subsequent pump unit.

Referring back to FIG. 4, 3 pumps may be programmed for a relay infusion as steps 1, 2 and 3. Once relay step 1 (pump 1 402a) completes an infusion and transfers infusion parameters to the next pump (pump 2 402b), it is removed out of the relay sequence 402. Pump 1 may be programmed as relay step 4. Similarly, when relay step 2 (pump 2 402b) is completed and is removed from the relay sequence 402, pump 2 may be added back to the relay sequence 402 as relay step 5.

In an important aspect, in order to instantiate the take-over mode on multiple pumps operating on the same infusion parameters, the target pump detection module 408e of the control system 406 may be configured to perform a number of logical checks before adding a pump to the relay sequence 402. For example, the target pump detection module 408e may be configured to retrieve and compare device specific information saved on memory 404g of each infusion pump in order to determine that a type of a target pump is same as a take-over relay infusion pump on the connected HUB. The target pump detection module 408e may also determine whether a take-over relay infusion is programmed on at least one pump within the connected HUB, and whether a target pump is loaded with the identical software version as the take-over relay infusion on the connected HUB.

Each infusion pump may be loaded with various drug libraries including protocols, rule sets, and/or pump configuration settings, thereby providing medication pick lists for caregivers to select a desired therapy, medication profiles including hard and soft limits, and pump configuration settings including but not limited to distal pressure occlusion limits, air-in-line limits, callback settings, backlight display settings, etc. The target pump detection module 408e may be configured to determine whether a target pump is loaded with the identical drug library as the take-over relay infusion on the connected HUB, whether a target pump is programmed with the identical care area as the take-over relay infusion on the connected HUB, whether a target pump is programmed with an identical drug as the take-over relay infusion on the connected HUB, and whether a target pump is programmed with the identical concentration as the take-over relay infusion on the connected HUB. In addition, the target pump detection module 408e may be configured to check whether a target pump is programmed with the identical medical coding modifier as the take-over relay infusion on the connected HUB, and whether a target pump is loaded with an identical syringe size and manufacturer as the take-over relay infusion on the connected HUB.

Each pump in the relay sequence 402 may be configured to denote a step number at all times, thereby being identifiable as a pump in relay sequence 402. In one aspect, when a relay step completed notification is generated by the notification module 408g on the active pump in the take-over relay sequence 402 and there is at least one inactive relay pump detected by the target pump detection module 408e, the notification module 408g of the control system 406 may be configured to notify the user the relay step infusion has completed, allow the user to review the completed relay step infusion information, and remove the completed pump from the relay sequence. The parameter transfer module 408f may be configured to generate and transmit instructions to the relay sequence 402, such that the current infusion parameters of a completed infusion pump are transferred to the next relay step in the relay sequence 402. Moreover, the parameter transfer module 408f may determine whether the next relay step pump in the relay sequence 402 obtains the infusion parameters on. In response to detecting that all infusion parameters have been accurately transferred from the completed pump to the next, the configuration module 408d may generate and transmit instructions to the relay sequence 402 to start the next relay step infusion on the next relay step pump in the sequence.

In one embodiment, the parameter transfer module 408f may determine the following programming information to be transferred from an active relay step pump to the next pump in the take-over relay sequence 402 when the active relay pump has completed the take-over relay step infusion: a flow rate, a dose of the drug (when applicable), a drug amount (when applicable), patient weight (when applicable), a patient's body surface area (BSA) (when applicable), occlusion setting, alarm volume setting, phase complete setting, pressure indicator setting, amount given, auto-brightness setting, brightness setting, keypad backlight setting, air in line setting, and near empty setting. It should be appreciated that the parameter transfer module 408f may determine any relevant operational parameters to be transferred among different pumps of the take-over relay sequence 402 to ensure that the next pump is replicating the previous pump and continuing the infusion without any interruption.

In another aspect, the control system 406 may generate alarms specific to interruption of the relay sequence 402, thereby mitigating risks of interruptions during an infusion therapy. For example, in response to detecting that the infusion on an active pump is completed, the control system 406 may automatically transfer all the programmed parameters from the completed step to the next pump and start the infusion on the next pump in sequence without generating an alarm signal to the user. A notification may be generated by the control system 406 when the relay step is completed. As a result, the user will have minimal interaction with each individual infusion pump 402a, 402b, 402c . . . 402n of the relay sequence 402 during an auto-continuous infusion therapy to a patient.

Based on the foregoing, the present disclosure advantageously reduces a user's alarm fatigue because multiple infusions are combined into one relay sequence. As a result, there may be no infusion complete alarm for each individual pump of a relay sequence during an auto-continuous infusion therapy except the last pump of the relay sequence, and no bag or syringe empty alarm. The present disclosure provides a seamless experience to a user. For example, referring to FIG. 1, a series of infusion pumps are pre-programmed and each pump of a relay sequence starts automatically in accordance with customized programming of the relay sequence. Further, referring to FIG. 4, the user may only be required to fully program one pump of a relay sequence and all relevant operational parameters of a completed pump are transferred to next pump in the sequence automatically. Thus, there is no need to constantly monitor or calculate time remaining for each infusion pump in a relay sequence, thereby providing the user with the flexibility to leave a patient's bedside, while the patient is receiving prescribed infusion medications on time.

According to aspects of the present disclosure, FIG. 6 illustrates a flowchart of a computer-implemented method 600 for establishing (602) a relay sequence of a plurality of infusion pumps to sequentially deliver drugs associated with each of the plurality of infusion pumps to a patient, wherein the plurality of infusion pumps include a first infusion pump and a second infusion pump. In response to detecting that the first infusion pump completes an infusion, method 600 comprises automatically transferring (604) infusion status information of the first infusion pump to the second infusion pump in the relay sequence; activating (606) the second infusion pump to start infusing in accordance with the relay sequence; removing (608) the first infusion pump from the relay sequence; and retaining (610) the operational parameters of the first infusion pump.

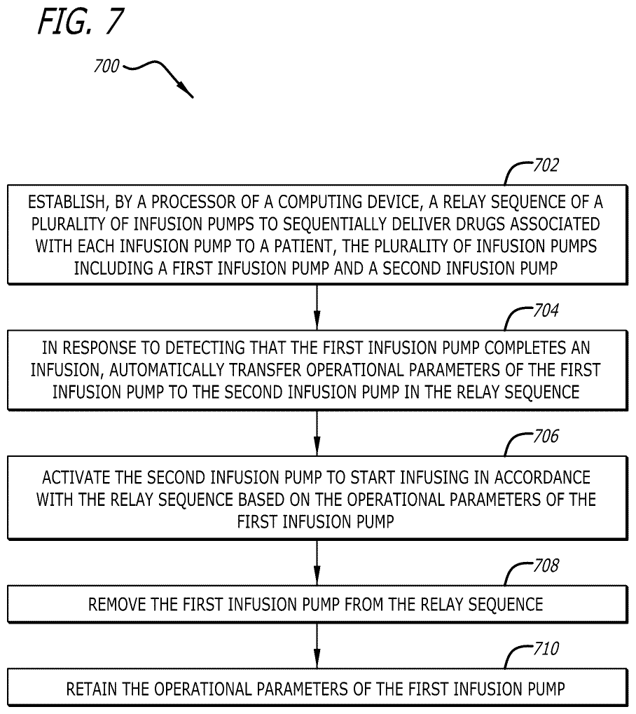

According to additional aspects of the present disclosure, FIG. 7 illustrates a flowchart of a computer-implemented method 700 for establishing (702) a relay sequence of a plurality of infusion pumps to sequentially deliver drugs associated with each of the plurality of infusion pumps to a patient, wherein the plurality of infusion pumps include a first infusion pump and a second infusion pump. In response to detecting that the first infusion pump completes an infusion, method 700 comprises automatically transferring (704) operational parameters of the first infusion pump to the second infusion pump in the relay sequence; activating (706) the second infusion pump to start infusing in accordance with the relay sequence based on the operational parameters of the first infusion pump; removing (708) the first infusion pump from the relay sequence; and retaining (710) the operational parameters of the first infusion pump.

The above description of the disclosure is provided to enable a person skilled in the art to make or use the disclosure. Various modifications to the disclosure will be readily apparent to those skilled in the art, and the common principles defined herein may be applied to other variations without departing from the spirit or scope of the disclosure. Further, the above description in connection with the drawings describes examples and does not represent the only examples that may be implemented or that are within the scope of the claims.

Furthermore, although elements of the described aspects and/or embodiments may be described or claimed in the singular, the plural is contemplated unless limitation to the singular is explicitly stated. Additionally, all or a portion of any aspect and/or embodiment may be utilized with all or a portion of any other aspect and/or embodiment, unless stated otherwise. Thus, the disclosure is not to be limited to the examples and designs described herein but is to be accorded the widest scope consistent with the principles and novel features disclosed herein.

1. A drug infusion system comprising:
a plurality of infusion pumps including a first infusion pump and a second infusion pump; and
a computing device, comprising:
a non-transitory computer-readable storage medium; and
a processor coupled to the non-transitory computer-readable storage medium and configured to control a plurality of modules to:
establish a relay sequence of the plurality of infusion pumps to sequentially deliver drugs associated with each of the plurality of infusion pumps to a patient;
in response to detecting that the first infusion pump completes an infusion, automatically transfer infusion status information of the first infusion pump to the second infusion pump in the relay sequence;
activate the second infusion pump to start infusing in accordance with the relay sequence;
remove the first infusion pump from the relay sequence; and
retain operational parameters of the first infusion pump.

2. The drug infusion system of claim 1, wherein the processor is further configured to control the plurality of modules to assign a relay step number for each of the plurality of infusion pumps in order to sequentially deliver the drugs associated with each of the plurality of infusion pumps to the patient.

3. The drug infusion system of claim 2, wherein the first infusion pump is configured to generate and transmit a relay step complete message to remaining infusion pumps of the relay sequence when the first infusion pump completes the infusion.

4. The drug infusion system of claim 3, wherein the processor is further configured to control the plurality of modules to reprogram the relay step number of the first infusion pump after the first infusion pump completes the infusion.

5. The drug infusion system of claim 1, wherein the processor is further configured to control the plurality of modules to add the first infusion pump back to the relay sequence after the first infusion pump completes the infusion based at least upon a reprogrammed relay step number of the first infusion pump.

6. The drug infusion system of claim 5, wherein the first infusion pump automatically operates in accordance with the reprogrammed relay step number using the operational parameters after being added back to the relay sequence.

7. The drug infusion system of claim 2, wherein the processor is further configured to control the plurality of modules to generate notification signals after each relay step is completed.

8. A computer-implemented method comprising:
establishing a relay sequence of a plurality of infusion pumps to sequentially deliver drugs associated with each of the plurality of infusion pumps to a patient, wherein the plurality of infusion pumps includes a first infusion pump and a second infusion pump;
in response to detecting that the first infusion pump completes an infusion, automatically transferring infusion status information of the first infusion pump to the second infusion pump in the relay sequence;
activating the second infusion pump to start infusing in accordance with the relay sequence;
removing the first infusion pump from the relay sequence; and
retaining operational parameters of the first infusion pump.

9. The computer-implemented method of claim 8, further comprising assigning a relay step number for each of the plurality of infusion pumps in order to sequentially deliver the drugs associated with each of the plurality of infusion pumps to the patient.

10. The computer-implemented method of claim 9, further comprising generating and transmitting, by the first infusion pump, a relay step complete message to remaining infusion pumps of the relay sequence when the first infusion pump completes the infusion.

11. The computer-implemented method of claim 10, further comprising reprogramming the relay step number of the first infusion pump after the first infusion pump completes the infusion.

12. The computer-implemented method of claim 8, further comprising adding the first infusion pump back to the relay sequence after the first infusion pump completes the infusion based at least upon a reprogrammed relay step number of the first infusion pump.

13. The computer-implemented method of claim 12, further comprising automatically operating the first infusion pump in accordance with the reprogrammed relay step number using the operational parameters after being added back to the relay sequence.

14. The computer-implemented method of claim 8, further comprising generating notification signals after each relay step is completed.

15. A non-transitory computer readable medium storing computer executable instructions for a system, the instructions being configured for:
establishing, by a processor of a computing device, a relay sequence of a plurality of infusion pumps to sequentially deliver drugs associated with each of the plurality of infusion pumps to a patient, wherein the plurality of infusion pumps include a first infusion pump and a second infusion pump;
in response to detecting that the first infusion pump completes an infusion, automatically transferring infusion status information of the first infusion pump to the second infusion pump in the relay sequence;
activating the second infusion pump to start infusing in accordance with the relay sequence;
removing the first infusion pump from the relay sequence; and
retaining operational parameters of the first infusion pump.

16. The non-transitory computer readable medium of claim 15, further comprising instructions for:
assigning a relay step number for each of the plurality of infusion pumps in order to sequentially deliver the drugs associated with each of the plurality of infusion pumps to the patient; and
generating notification signals after each relay step is completed.

17. The non-transitory computer readable medium of claim 16, further comprising instructions for generating and transmitting, by the first infusion pump, a relay step complete message to remaining infusion pumps of the relay sequence when the first infusion pump completes the infusion.

18. The non-transitory computer readable medium of claim 17, further comprising instructions for reprogramming the relay step number of the first infusion pump after the first infusion pump completes the infusion.

19. The non-transitory computer readable medium of claim 18, further comprising instructions for adding the first infusion pump back to the relay sequence after the first infusion pump completes the infusion based at least upon a reprogrammed relay step number of the first infusion pump.

20. The non-transitory computer readable medium of claim 19, further comprising instructions for automatically operating the first infusion pump in accordance with the reprogrammed relay step number using the operational parameters after being added back to the relay sequence.
